(19) United States
(12) Patent Application Publication
(10) Pub. No.: US 2025/0205475 A1
(43) Pub. Date: Jun. 26, 2025

(54) CONTROL UNIT FOR A BLOOD PUMP, PUMP SYSTEM AND METHOD

(71) Applicant: Berlin Heart GmbH, Berlin (DE)

(72) Inventors: Oliver Peters, Berlin (DE); Matthias Kiesner, Rangsdorf (DE); Robert Steingräber, Berlin (DE)

(73) Assignee: Berlin Heart GmbH, Berlin (DE)

(21) Appl. No.: 18/849,218

(22) PCT Filed: Mar. 21, 2023
(86) PCT No.: PCT/EP2023/057219

§ 371 Date: Sep. 20, 2024

(30) Foreign Application Priority Data
Mar. 21, 2022 (EP) 22163254.0

(51) Int. Cl. A61M 60/538; A61M 60/178; A61M 60/216; A61M 60/422; A61M 60/82

(52) U.S. Cl. CPC A61M 60/538; A61M 60/178; A61M 60/216; A61M 60/422; A61M 60/82; A61M 2205/3365

(57) ABSTRACT

A control unit for a blood pump, a pump system comprising such a control unit, and a method for controlling a blood pump may be provided. A control unit may be configured to determine a control signal on the basis of a measurement signal, in such a way that a rotor of a blood pump is supported contact-free in a housing and a rotation of the rotor is closed-loop-controlled, where a specification for a rate of change of the rotation speed of the rotation of the rotor about the rotation axis may be limited upwardly by a predefined maximum rate of change, and to control the stator in accordance with the determined control signal.

### CROSS-REFERENCE TO RELATED APPLICATIONS

This application is a U.S. national stage application under 35 U.S.C. § 371 of PCT Application No. PCT/EP2023/057219, filed on Mar. 21, 2023, and published on Sep. 28, 2023 as WO 2023/180325 A1, which claims priority to European Patent Office Application No. EP 22163254.0, filed on Mar. 21, 2022. The entire contents of each of the above applications is incorporated herein by reference.

### BRIEF DESCRIPTION OF THE DRAWINGS

Exemplary embodiments of the subject matter of the application are explained below with reference to drawings. The drawings show:

FIG. 1 a schematic representation of a pump system with a blood pump and a control unit,

FIG. 2 time curves of a simulated measurement signal in various operating states of a pump system,

FIG. 3 a schematic representation of a pump control,

FIG. 4a and FIG. 4b schematic illustrations of a vector decomposition of a back-EMF,

FIG. 5 a schematic illustration of a pump control according to a further example.

Recurring and similar features of different embodiments are provided with identical alphanumeric reference signs in the illustrations.

### DETAILED DESCRIPTION

The application relates to a control unit for a blood pump, in particular a rotary fluid pump for cardiac support, a pump system comprising such a control unit and a method for controlling a blood pump.

A blood pump controllable by means of a control unit comprises a rotor magnetically supported in a housing and rotatable about a rotation axis for conveying a fluid, and a stator, wherein the stator is configured to generate a variable stator magnetic field for exerting a variable bearing force along a bearing direction of action and a torque about the rotation axis on the rotor, wherein the blood pump is configured to provide a measurement signal which depends on a position and/or movement of the rotor in the housing.

Blood pumps of this type as well as corresponding control units and control methods are known from the prior art.

When controlling such a blood pump, it may be provided to determine, on the basis of the measurement signal, a control signal for varying the stator magnetic field, in such a way that the rotor is supported in the housing contact-free along the bearing direction of action by means of the bearing force and a rotation of the rotor about the rotation axis is generated by means of the torque.

Various influences may occur that complicate the steps required to determine a control signal for controlling the contact-free bearing and the rotation of the rotor. These include for example the inherently complex dynamics of the blood pump as a system to be controlled, instabilities of this system, systematic and stochastic measurement errors as well as possible couplings of different measurement parameters and/or system degrees of freedom. If, for example, rotation-dependent and translation-dependent components of the measurement signal are unable to be clearly separated when evaluating the measurement signal, this may impair the reliability of a closed-loop control for stabilizing rotational and translational degrees of freedom.

Against the background of the prior art, the application addresses the problem of providing a control unit for a blood pump, a pump system and a method for controlling a blood pump, which reduce or avoid the problems mentioned and in particular enable reliable and safe operation of the blood pump with regard to closed-loop control of the contact-free bearing and the rotation of the rotor.

To solve the problem, control units, pump systems, and methods are proposed. Advantageous embodiments and further developments are described in conjunction with the features described in the application.

A proposed control unit is configured for controlling a blood pump of the above-mentioned type, wherein the measurement signal comprises a component dependent on a bearing position and/or bearing speed of a movement of the rotor along the bearing direction of action (hereinafter also translation-dependent component of the measurement signal) and a component dependent on a rotation angle and/or a rotation speed of a rotation of the rotor about the rotation axis (hereinafter also rotation-dependent component of the measurement signal). The measurement signal may be a one- or multi-dimensional measurement signal, for example, it may comprise a single sensor signal or a plurality of similar or different sensor signals and/or signals derived from them.

The control signal is typically a multi-dimensional signal. If the bearing force and the torque are generated by means of substantially independent actuators or actuator components, a rotary component of the control signal and a translational component of the control signal may correspond to different vector components of the control signal. If, on the other hand, bearing force and torque are generated by means of coupled actuators or actuator components, which therefore influence both the bearing force and the torque, one or more vector components of the control signal may each comprise both a translational component and a rotary component.

The blood pump is preferably configured to convey the fluid, in particular blood, from an inlet to an outlet. The inlet and outlet may each be connected directly or indirectly, in particular by means of a cannula, to a heart and/or a blood vessel. The blood pump may be used in particular as a VAD (ventricular assist device).

The control unit is configured to determine, on the basis of the measurement signal, a control signal for varying the stator magnetic field, in such a way that the rotor is supported in the housing contact-free along the bearing direction of action by means of the bearing force and, by means of the torque, a rotation of the rotor about the rotation axis is generated, in particular open-loop- and/or closed-loop-controlled, wherein a specification for a rate of change of the rotation speed of the rotation of the rotor about the rotation axis is limited upwardly by a predefined maximum rate of change, such as to limit an influence of a change in the rotation speed on the determined control signal.

The control signal may comprise a translational component for contact-free supporting of the rotor along the bearing direction of action in the housing and a rotary component for controlling the rotation of the rotor.

The fact that the specification for the rate of change of the rotation speed is limited such as to limit the influence of the change of the rotation speed on the determined control signal is to be understood in such a way that a dependency of the control signal on the change of the rotation speed is at least temporarily lower than when the control signal is determined without limitation of the rate of change and/or in such a way that a maximum dependency of the control signal on the change of the rotation speed is lower than when the control signal is determined without limitation of the rate of change.

By limiting the specification for the rate of change of the rotation speed in the aforementioned manner, it is possible to dynamically decouple the rotary component of the control signal and the translational component of the control signal, at least partially, so that the rotor is positioned along the bearing direction of action comparatively quickly and the rotor rotation is controlled comparatively slowly. This improves the reliability and safety of the blood pump with regard to the control of the contact-free bearing and the rotation of the rotor. This applies in particular if, for example, rotation-dependent and translation-dependent components of the measurement signal are unable to be clearly separated from each other when evaluating said measurement signal, which for example may be the case if several different system states of the blood pump may lead to identical or, in any case, indistinguishable or almost distinguishable values and/or time courses of the measurement signal within the scope of the measurement accuracy. Such a system is referred to below as an ambiguous system. The control unit is also configured to control the stator in accordance with the determined control signal. The control unit is preferably configured to carry out a detection of the measurement signal, the determination of the control signal and the corresponding control of the stator several times repeatedly in a sequence of control cycles.

The predefined maximum rate of change can be selected in particular such that the influence of the change in the rotation speed by the control of the stator in accordance with the determined control signal on the control signal, in particular on a determination of the bearing position and/or bearing speed performed on the basis of the measurement signal and contributing to the control signal is limited compared to control of the stator in accordance with a control signal that is determinable without limitation of the rate of change of the rotation speed to the predefined maximum rate of change.

The control unit is preferably configured to determine the control signal in such a way that a predefined and/or actual and/or measured rate of change of the rotation speed of the rotor does not reach or exceed the specification and/or the predefined maximum rate of change when the stator is controlled in accordance with the determined control signal.

In particular, the predefined maximum rate of change may be a parameter stored or storable within the control unit, in particular an open-loop or closed-loop control parameter. The predefined maximum rate of change may be a factory-set specification and/or a specification that is unchangeable during operation. The control unit may be configured to determine the predefined maximum rate of change on the basis of one or more operating parameters of the blood pump. The control unit may be configured to redetermine the predefined maximum rate of change at certain intervals, for example in each control cycle or after a specified number of control cycles or several times in a control cycle. The predefined maximum rate of change may alternatively or additionally be given by one or more design properties, for example mechanical, electrical and/or magnetic properties, of the blood pump, for example by a mass inertia of the rotor and/or a maximum achievable motor torque.

The predefined maximum rate of change (exemplary unit rad/s2) may be proportional to a dynamic parameter of the movement of the rotor along the bearing direction of action. The dynamic parameter may be a parameter of an open-loop or closed-loop control of the movement of the rotor along the bearing direction of action and/or a variable determined on the basis of the measurement signal. For example, the dynamic parameter is a bearing acceleration and/or deflection frequency and/or deflection frequency rate of change of the movement of the rotor along the bearing direction of action and/or a gain crossover frequency and/or resonance frequency of a closed-loop control of the movement of the rotor along the bearing direction of action.

A proportionality constant, which links the predefined maximum rate of change and the dynamic parameter, may be selected in such a way that the movement of the rotor along the bearing direction of action is normalized to a “rotation period”, i.e. to a full circle angle, taking into account a maximum deflection range of the rotor along the bearing direction of action, so that the predefined maximum rate of change and the predefined dynamic parameter are comparable with each other in identical units. Thus, for example, the bearing acceleration may be represented in the units of the rate of change of the rotation speed (e.g. rad/s2).

The predefined maximum rate of change may be given by a time constant, in particular a delay time and/or step response time and/or rise time, of a control of the rotation of the rotor. For example, the rotation speed of the rotor may be controlled using a slow controller, such as a controller without a D element. Then the rotation speed of the rotor is unable to change faster than a time constant, such as the step response time, of the controller allows, whereby the predefined maximum rate of change is given by this time constant and the specification for the rate of change of the rotation speed of the rotation of the rotor is correspondingly limited upwards.

It may be provided that the predefined maximum rate of change is at most half, preferably at most one fifth, in particular at most one tenth of a characteristic rate of change, such as the rate of change of the deflection frequency or the bearing acceleration, of the movement of the rotor along the bearing direction of action. It may be provided that a characteristic time constant, by which the predefined maximum rate of change is given, is at least twice, preferably five times, in particular at least ten times a characteristic time constant of the movement of the rotor along the bearing direction of action.

To illustrate possible variables of the system dynamics, some possible values of the predefined maximum rate of change and various dynamic parameters are given below. The specified values and value ranges are to be understood as examples; deviating values and value ranges are possible. A rotation speed of the rotor (hereinafter also referred to as rotor speed or rotation speed) may be variable in the range from 1000 to 4000 rpm, for example. A rotor speed of 4000 rpm corresponds to approximately 420 rad/s or 66 Hz. The maximum rate of change may be in the range of 100 rpm/ms to 1000 rpm/ms, for example. A preferred maximum rate of change of 400 rpm/ms corresponds to approximately 42000 rad/s2. The bearing speed may be in particular in the range of 0.1 m/s to 1 m/s, for example approximately 0.5 m/s. The bearing acceleration may be in particular in the range of 50 m/s2 to 1000 m/s2, for example approximately 500 m/s2. For example, a value of less than 50 Hz, for example approximately 20 Hz, may be provided as the limit frequency for controlling the rotation rate of change. For example, a value of more than 50 Hz, for example approximately 100 Hz, may be provided as the limit frequency for controlling the bearing acceleration.

The measurement signal may be a one- or multi-dimensional measurement signal, for example, it may comprise a single sensor signal or a plurality of similar or different sensor signals and/or signals derived from them. The measurement signal may correspond to a back-electromotive force (back-EMF) induced on account of a magnetic field change in the stator caused by the rotation of the rotor and/or the movement of the rotor along the bearing direction of action. The measurement signal corresponding to the back-EMF may be detected without an additional sensor by means of the motor coil windings of the stator, which therefore enables “sensorless” generation of the measurement signal. Controlling a system using a controller based on sensorless generation of a measurement signal is also referred to as sensorless control and is used in particular for brushless DC motors (BLDC motors). The rotor and stator of the blood pump may form a BLDC motor.

A difference between sensorless control in the sense of this application and sensorless control of BLDC motors according to the prior art should be discussed here. A sensorless commutation is known from the prior art of sensorless control of BLDC motors. The rotation angle of the rotor is determined here sensor-free on the basis of the back-EMF, and an electrical commutator or field-oriented control generates the necessary phase signals to rotate the motor. In conventional motors, which are supported using ball bearings, for example, the magnetic flux generated by the rotor magnets in the stator is constant to a first approximation or changes only slowly with the temperature of the rotor magnets. In an axial flux motor, the magnetic flux is also dependent on the axial rotor position (bearing position). If the axial rotor position is variable here, then the axial position may be deduced from the magnetic flux. In the case of “sensorless” detection based on back-EMF within the meaning of this application, the change in the magnetic flux generated by the rotor, which flows through the stator coils, is determined over time (the stator coils are therefore used as a sensor). Here, a rotational oscillation may generate a similar induction signal as an axial translational oscillation, which means that the system may be ambiguous.

The use of the back-EMF to commutate a VAD is shown, for example, in CA2687114C and EP3827852A1. According to the latter document (paragraph 19, lines 49-52), the back-EMF in an axial flux motor is influenced by both the axial rotor speed (bearing speed) and the axial rotor position. The back-EMF is amplitude-modulated quasi-statically by the rotor position.

However, a highly dynamic change in the axial rotor position, for example within less than 5 ms, results in further components of the back-EMF in addition to the pure amplitude modulation. As the inventors of the present application have recognized, these components distort both the amplitude and the phase of the three-phase back-EMF. The subject matter of the present application is particularly suitable for reducing, compensating or correcting these components.

When using a sensorless measurement signal, it may therefore be particularly difficult to separate the rotation-dependent and translation-dependent components of the measurement signal. The dynamic decoupling of the rotary and translational components of the control signal as described above may therefore improve the operation of a sensorless blood pump and thus also contribute to a particularly compact and robust design of the blood pump. Furthermore, the number of wires that must be provided in a driveline for transmitting the measurement signal is particularly low when the measurement signal is generated without sensors, which may also enable a robust and compact design and/or simplified provision of redundancy.

It may be provided that a plurality of measurement points, corresponding to the measurement signal at a plurality of time points, is detected and the control unit is configured to determine the control signal on the basis of the plurality of measurement points. In this way, a time curve of the measurement signal is taken into account over a number of points in time when determining the control signal, which may help to reduce ambiguity, particularly in the case of an ambiguous system, and thus enable more reliable control of the blood pump. The measurement of a measurement point at a corresponding time point is also referred to as a time step or sampling step, while the time interval between two measuring points is referred to as the sampling time.

The control unit may be configured to estimate, on the basis of the measurement signal, one or more state parameters of a model of the movement of the rotor, in particular the rotation of the rotor about the rotation axis and/or the movement of the rotor along the bearing direction of action, wherein the state parameter or state parameters comprise in particular the rotation angle and/or the rotation speed and/or the rate of change of the rotation speed and/or a rotation frequency and/or a rotation frequency rate of change of the rotation of the rotor and/or the bearing position and/or the bearing speed and/or a bearing acceleration and/or a deflection frequency and/or a deflection frequency rate of change of the movement of the rotor along the bearing direction of action and/or the bearing force and/or the torque and/or a field strength of a magnetic field generated within the stator by the rotor and/or a change of this field strength over time, and to determine the control signal on the basis of one or more of the estimated state parameters. The estimation of the state parameter or state parameters of the model is carried out by means of an observer or by observation (in the sense of control technology) and may be carried out in various ways.

For example, the control unit may be configured to estimate the one or more status parameters using an Extended Kalman Filter (EKF). An EKF is particularly suitable for observing a non-linear dynamic system.

It may be provided that the state parameter or state parameters comprise a torque amplification factor, which corresponds to a ratio of the rotation speed and the torque. Consideration of this state parameter may be advantageous for the design of the observer, as illustrated below using examples.

The control unit may be configured to estimate the one or more status parameters using a manually interpreted observer. Manual interpretation may reduce the computational effort required for observation compared to an estimation method such as the EKF.

The control unit may be configured to estimate the one or more state parameters using a vector decomposition of a measured back-EMF into a rotation component and a bearing position component, which enables a particularly simple estimation, as explained in more detail below.

The control unit may be configured to receive a sensor signal provided using a rotor position sensor of the blood pump, depending on the bearing position and/or bearing speed of the movement of the rotor along the bearing direction of action. The control unit may then further be configured to determine, on the basis of the sensor signal and the measurement signal, a corrected measurement signal which corresponds to the component of the measurement signal dependent on the rotation angle and/or the rotation speed of the rotation of the rotor about the rotation axis. Furthermore, the control unit may be configured to determine a rotary component of the control signal (as defined above) on the basis of the corrected measurement signal. The control unit may be configured to determine a translational component of the control signal (as defined above) on the basis of the sensor signal.

By determining the rotary or translational component of the control signal in the manner described above, further decoupling of these components may be achieved. This further improves the reliability and safety of the blood pump with regard to the control of the contact-free bearing and the rotation of the rotor.

A further proposed control unit is configured to control a blood pump of the above-mentioned type, wherein the blood pump further comprises a rotor position sensor configured to provide a position sensor signal dependent on the bearing position and/or bearing speed of the movement of the rotor along the bearing direction of action. It is again provided that the measurement signal comprises a component dependent on a bearing position and/or bearing speed of a movement of the rotor along the bearing direction of action (hereinafter also translation-dependent component of the measurement signal) and a component dependent on a rotation angle and/or a rotation speed of a rotation of the rotor about the rotation axis (hereinafter also rotation-dependent component of the measurement signal). The further proposed control unit is configured to determine a corrected measurement signal on the basis of the position sensor signal and the measurement signal, which corrected measurement signal corresponds to the component of the measurement signal dependent on the rotation angle and/or the rotation speed of the rotation of the rotor about the rotation axis, to determine a control signal on the basis of the measurement signal for varying the stator magnetic field in such a way that the rotor is supported in the housing contact-free along the bearing direction of action by means of the bearing force and a rotation of the rotor about the rotation axis is generated, in particular open-loop- and/or closed-loop-controlled, by means of the torque, to determine a corrected measurement signal on the basis of the sensor signal and the measurement signal, which corrected measurement signal corresponds to the component of the measurement signal dependent on the rotation angle and/or the rotation speed of the rotation of the rotor about the rotation axis, wherein a rotary component of the control signal for closed-loop control of the rotation of the rotor is determined on the basis of the corrected measurement signal, and to control the stator in accordance with the determined control signal.

By determining the rotary or translational component of the control signal in the manner described above, a decoupling of these components may in turn be achieved. This improves the reliability and safety of the blood pump with regard to the control of the contact-free bearing and the rotation of the rotor.

With regard to the further or optional features described below, “control unit” may refer to a proposed control unit of the first type described above or to a control unit of the second type described above (i.e. the further proposed control unit).

The control unit may be configured to determine the control signal in such a way that the rotor is adjusted into a target bearing position at which external forces acting on the rotor along the bearing direction of action add up to a predetermined force, in particular a zero force, and/or at which a power applied to generate the variable stator magnetic field is minimal.

The control unit may also be configured to detect a plurality of measurement points, corresponding to at least a component of the measurement signal at a plurality of time points with known, in particular constant rotation speed and constant bearing position of the rotor, to fit a periodic correction function, in particular a sine function or a plurality of superposed sine functions, to at least one component of the measurement signal by means of optimization.

On the basis of the correction function fitted in this way, a disturbance of the measurement signal that depends on the rotation angle of the rotor, in particular one that occurs periodically with a period of rotation of the rotor, may be compensated for and thus its influence on the control signal may be reduced. Such a disturbance may occur, for example, due to asymmetries in the system, such as the rotor magnetic field and/or the motor coils.

The proposed pump system comprises a blood pump and a control unit of the proposed type, configured to control the blood pump, wherein the blood pump comprises a rotor magnetically supported in a housing and rotatable about a rotation axis for conveying a fluid, and a stator, wherein the stator is configured to generate a variable stator magnetic field for exerting on the rotor a variable bearing force along a bearing direction of action and a torque about the rotation axis; wherein the blood pump is configured to provide a measurement signal comprising a component dependent on a bearing position and/or speed of a movement of the rotor along the bearing direction of action and a component dependent on a rotation angle and/or a rotation speed of a rotation of the rotor about the rotation axis.

In the proposed pump system, the proposed control unit provides its advantages described above.

The control device is suitable for various types of blood pumps, which may differ, for example, in the type of electric motor formed by the rotor and stator and/or the bearing direction of action. The rotor and the stator may, for example, form an axial flux motor or radial flux motor, wherein the bearing direction of action is at a non-zero angle, in particular substantially perpendicular to the radial direction of the rotation axis (X) of the rotor (320). In addition to the aforementioned bearing direction of action, the rotor (320) may also be actively magnetically supported in the housing (301) along a second bearing direction of action.

The rotor may generate a non-linear, in particular at least approximately exponentially decaying, permanent magnetic field along the bearing direction of action and/or the rotation axis.

A proposed method is intended for controlling a blood pump, wherein the blood pump comprises a rotor magnetically supported in a housing and rotatable about a rotation axis for conveying a fluid, and a stator, wherein the stator is configured to generate a variable stator magnetic field for exerting a variable bearing force along a bearing direction of action; wherein the blood pump is configured to provide a measurement signal comprising a component dependent on a bearing position and/or speed of a movement of the rotor along the bearing direction of action and a component dependent on a rotation angle and/or a rotation speed of a rotation of the rotor about the rotation axis.

The method comprises: determining, on the basis of the measurement signal, a control signal for varying the stator magnetic field, in such a way that the rotor is supported in the housing contact-free along the bearing direction of action by means of the bearing force and, by means of the torque, a rotation of the rotor about the rotation axis is generated, in particular open-loop- and/or closed-loop-controlled, wherein a specification for a rate of change of the rotation speed of the rotation of the rotor about the rotation axis is limited upwardly by a predefined maximum rate of change, such as to limit an influence of a change in the rotation speed on the determined control signal, and controlling the stator in accordance with the determined control signal.

A further proposed method is intended for controlling a blood pump, wherein the blood pump comprises a rotor magnetically supported in a housing and rotatable about a rotation axis for conveying a fluid, and a stator, wherein the stator is configured to generate a variable stator magnetic field for exerting a variable bearing force along a bearing direction of action along the bearing direction of action; wherein the blood pump is configured to provide a measurement signal comprising a component dependent on a bearing position and/or speed of a movement of the rotor along the bearing direction of action and a component dependent on a rotation angle and/or a rotation speed of a rotation of the rotor about the rotation axis; wherein the blood pump comprises a rotor position sensor which is configured to provide a position sensor signal dependent on the bearing position and/or bearing speed of the movement of the rotor along the bearing direction of action.

The method comprises: determining, on the basis of the position sensor signal and the measurement signal, a corrected measurement signal which corresponds to the component of the measurement signal dependent on the rotation angle and/or the rotation speed of the rotation of the rotor about the rotation axis, determining, on the basis of the corrected measurement signal, a control signal for varying the stator magnetic field, in such a way that the rotor is supported in the housing contact-free along the bearing direction of action by means of the bearing force and, by means of the torque, a rotation of the rotor about the rotation axis is generated, in particular open-loop- and/or closed-loop-controlled, and determining, on the basis of the sensor signal and the measurement signal, a corrected measurement signal which corresponds to the component of the measurement signal dependent on the rotation angle and/or the rotation speed of the rotation of the rotor about the rotation axis, wherein a rotary component of the control signal for closed-loop control of the rotation of the rotor on the basis of the corrected measurement signal is determined, and controlling the stator in accordance with the determined control signal.

In particular, the proposed methods may be carried out using a control unit of the type proposed above and/or a pump system of the type proposed above and may be further embodied in accordance with the optional features of the control unit and the pump system.

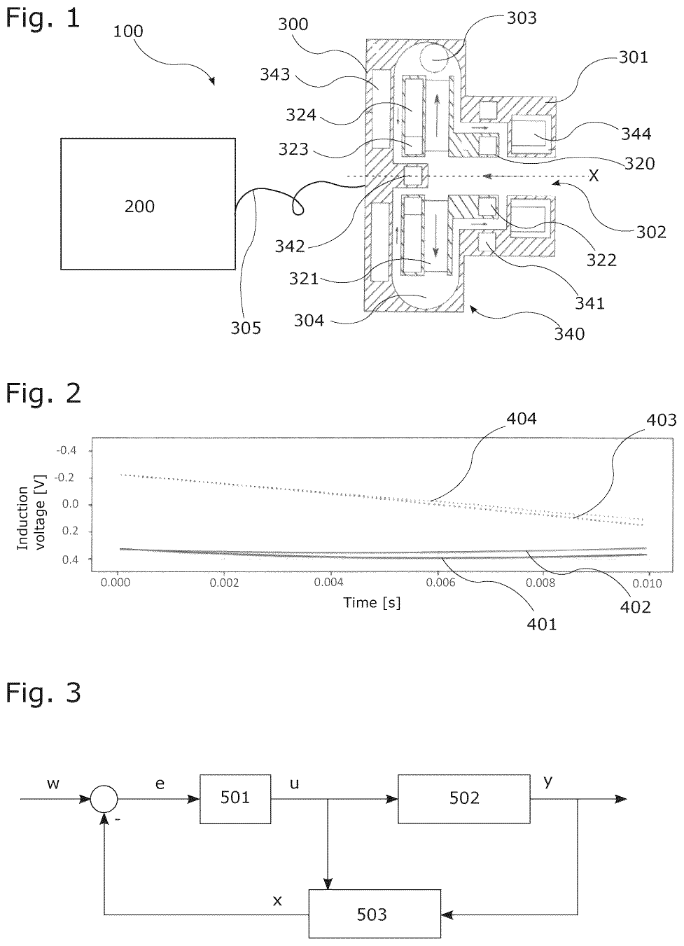

The pump system 100 shown in FIG. 1 comprises a blood pump 300 and a control unit 200, wherein the control unit 200 is configured to control the blood pump 300.

The blood pump 300 is formed as a rotary fluid pump for cardiac support and comprises a rotor 320 magnetically supported in a housing 301 and rotatable about a rotation axis X for conveying a fluid, in particular blood, from an inlet 302 to an outlet 303, and a stator 340 formed in the housing 301.

In the example shown, the inlet 302 is connectable directly (by implantation in a heart wall) to a ventricle of a heart; the outlet 303 is connectable by means of a cannula to a blood vessel connected to the heart. The blood pump may therefore be used as a VAD.

The rotor 320 and the stator 340 of the blood pump 340 form a three-phase BLDC motor. The stator 340 comprises a motor coil arrangement 343, the rotor 320 comprises a motor magnet arrangement 324, so that by energizing the motor coils a variable stator magnetic field may be generated for exerting a variable bearing force along a bearing direction of action and a torque with respect to the rotation axis X on the rotor 320.

The bearing direction of action is substantially parallel to the rotation axis X and is also referred to below as the axial direction. Perpendicular directions are therefore referred to as radial or lateral directions. The bearing direction of action may be arranged in a direction other than the axial direction, for example in the radial or lateral direction or in a direction at a different angle to the rotation axis X.

The rotor 320 is therefore actively magnetically supported in the housing 301 along the bearing direction of action by means of the variable bearing force. The motor coil arrangement 343 and the motor magnet arrangement 324 act as bearing magnets. Additionally or alternatively, the stator 340 may comprise at least one optional control coil 344 which acts to exert the bearing force on at least one magnet of the rotor 320, in the example shown on the inlet-side rotor magnet bearing 323. The bearing control signal corresponds to a voltage to be applied to the motor coil arrangement 343 and/or the control coil 344.

In operation, the fluid enters the housing 301 and the rotor 320 substantially in the axial direction and is conveyed by a blading 321 in the radial direction into a volute 304 of the housing 301, from where it reaches the outlet 303. The blood flow is schematically illustrated by arrows in FIG. 1.

The blood pump 300 of the example shown is therefore embodied as a radial or centrifugal pump. However, the subject matter of the application is not limited to this type of pump, but is applicable to various types of rotary fluid pumps. For example, the blood pump may be an axial pump. The control unit 320 is also suitable for various pumps in terms of the type of electric motor formed by the rotor 320 and stator 340. In the example shown, the rotor 320 and the stator 340 form an axial flux motor, wherein the bearing direction of action substantially corresponds to the axial direction. Alternatively, the rotor and the stator may form a radial flux motor, wherein the bearing direction of action substantially corresponds to the radial direction.

The rotor 320 is magnetically supported in the housing 301 in all spatial directions. The rotor 320 is passively magnetically supported in the radial direction. To this end, the rotor 320 has an inlet-side rotor magnet bearing 322 and an outlet-side rotor magnet bearing 323, and the stator 340 has an inlet-side stator magnet bearing 341 and an outlet-side stator magnet bearing 342, with the inlet-side stator magnet bearing 341 acting on the inlet-side rotor magnet bearing 322 and the outlet-side stator magnet bearing 342 acting on the outlet-side rotor magnet bearing 323. As described above, the rotor 321 is actively magnetically supported in the axial direction. However, the subject matter of the application is not limited to the bearing geometry described, but may be applied to various pumps with an active magnetic bearing along at least one bearing direction of action.

The blood pump 300 is configured to provide a measurement signal which comprises a (translation-dependent) component dependent on a bearing position and/or speed of a movement of the rotor 320 along the bearing direction of action and a (rotation-dependent) component dependent on a rotation angle and/or a rotation speed of a rotation of the rotor 320 about the rotation axis X. The measurement signal corresponds to a back-electromotive force (back-EMF) induced on account of a magnetic field change in the stator 340 caused by the rotation of the rotor 320 and/or the movement of the rotor 320 along the bearing direction of action, that is to say is generated sensor-free. The measurement signal is a multi-dimensional (vectorial) measurement signal, the components of which correspond to the induction voltages in the three phases of the motor coil arrangement 343.

The blood pump 300 may alternatively or additionally comprise a sensor, for example an eddy current sensor and/or a magnetic field sensor, in particular a Hall sensor, to provide the measurement signal.

The blood pump 300 may comprise a rotor position sensor which is configured to provide a position sensor signal dependent on the bearing position and/or bearing speed of the movement of the rotor 320 along the bearing direction of action.

The back-EMF may be represented as follows using the law of induction:

$${V}_{BEMF} = - \frac{d φ}{dt} N = - \frac{dB}{dt} AN . \qquad ( equation 1 )$$

Here, VBEMF is the back-EMF, the voltage induced in the motor coil arrangement 343 by changing the magnetic field due to the movement of the rotor 320, ω is a magnetic flux, t is the time, N is a number of turns of a coil of the motor coil arrangement 343, B is a magnetic flux density, and A is an area of the coil.

The induction voltage V_{BEMF} induced by the movement of the rotor magnets 322, 323, 324 in a motor coil of the motor coil arrangement 343 is superimposed at the terminals of the coil by a resistive component V_{R}, a self-induction voltage V_{Lii} of the coil and a counter-induction voltage V_{Lij} for each additional motor coil. The sum V_{coil,i} IS measurable as phase voltage at the terminals of a single motor coil.

$${V}_{Coil} = {V}_{BEMF} + {V}_{R} + {V}_{Lii} + {\sum }_{j = 0}^{n} {V}_{Lij} . \qquad ( equation 2 )$$

The superimposed components may be determined from the phase currents Iᵢ:

$${V}_{R} = {I}_{i} {R}_{i} , \qquad ( equation 3 a )$$

$${V}_{Lii} = {L}_{i} \frac{{dI}_{i}}{dt} , \qquad ( equation 3 b )$$

$${V}_{Lij} = {L}_{ij} \frac{{dI}_{j}}{dt} . \qquad ( equation 3 c )$$

If the phase currents and phase voltages are known, the induction voltage V_{BEMF} may be calculated using equation 2. Only the sum of several phase voltages or phase currents may be measured on a three-phase motor in delta connection or star connection without star point terminal. In this case, the three sums of two phase voltages may be determined instead of the individual three phase voltages. This does not restrict the procedures described above.

Methods are known from the prior art which may determine the induction voltage with a reduced number of sensors. For example, the terminal voltage on the motor cables is estimated here using the duty cycle of a pulse width modulation and the supply voltage. Only two current measurements are then required to determine the induction voltages. For the special case Iᵢ=0, the induction voltage may be measured directly.

Since, of the three induction voltages {VA, VB, VC} in a three-phase motor connected with three cables, only two are independent as considered from the outside, these may be clearly converted to a two-phase system {Va, Vb} as follows using the Clarke transformation:

$$[ \begin{matrix}{V}_{a} \\ {V}_{b}\end{matrix} ] = \frac{2}{3} [ \begin{matrix}1 & - \frac{1}{2} & - \frac{1}{2} \\ 0 & \frac{\sqrt{3}}{2} & \frac{\sqrt{3}}{2}\end{matrix} ] [ \begin{matrix}{V}_{A} \\ {V}_{B} \\ {V}_{C}\end{matrix} ] . \qquad ( equation 4 )$$

FIG. 2 shows simulated measurement signal curves 401, 402, 403, 404 for an operation of the blood pump 300, wherein the measurement signal curves 401 and 402 correspond to the induction voltage Va, the measurement signal curves 403 and 404 correspond to the induction voltage Vb. The measurement signal curves 401 and 403 correspond to a first operating state in which the rotor rotates at a first speed about the rotation axis X and is in a first bearing position along the bearing direction of action. The measurement signal curves 402 and 404 correspond to a second operating state in which the rotor rotates about the rotation axis X at a second speed that is lower than the first speed and is located in a second bearing position that is displaced relative to the first bearing position, wherein an axial rotor movement is also present. The blood pump is operated here with a control cycle of 50 us duration, the time axis from 0 to 0.01 s thus corresponds to 200 control cycles.

As may be seen in FIG. 2, the same constellation of induction voltages (at time t=0) may be achieved by different combinations of bearing position and speed. For this reason, rotation-dependent and translation-dependent components of the measurement signal are unable to be clearly separated from each other when evaluating the aforementioned measurement signal; the system is therefore ambiguous. Although the values drift apart over time, it is not possible to wait for the necessary time delay of approximately 5 ms (25 control cycles) before deciding whether the torque or the bearing force needs to be adapted. During this time, the rotor 320 could already collide with a wall of the housing 301, which must be avoided at all costs during operation of the blood pump 300. The decision regarding the adaptation of torque or bearing force is therefore made by dynamically decoupling the rotary and translational components of the control signal as follows.

The control unit 200 is configured to determine, on the basis of the measurement signal, a control signal for varying the stator magnetic field, in such a way that the rotor 320 is supported in the housing 301 contact-free along the bearing direction of action by means of the bearing force and, by means of the torque, a rotation of the rotor 320 about the rotation axis X is controlled, wherein a specification for a rate of change of the rotation speed of the rotation of the rotor 320 (rotation speed rate of change) about the rotation axis is limited upwardly by a predefined maximum rate of change, such as to limit an influence of a change in the rotation speed on the determined control signal. The rotation of the rotor 320 does not have to be controlled in all cases; it is also possible to provide open-loop control of the rotation

The control signal is a multi-dimensional signal, corresponding to the phase voltages for controlling the BLDC motor. The control signal comprises a rotary component for contact-free bearing of the rotor 320 along the bearing direction of action in the housing 301 and a translational component for controlling the rotation of the rotor 320, wherein each vector component of the control signal contains both components in coupled form.

As explained above, by limiting the rotation speed rate of change, the rotary component of the control signal and the translational component of the control signal are dynamically partially decoupled, so that the supporting of the rotor 320 along the bearing direction of action is comparatively fast, the control of the rotation of the rotor is comparatively slow, and a reliability and safety of the blood pump 300 with respect to the control of the contact-free bearing and the rotation of the rotor 320 may be improved.

The control unit 200 is also configured to control the stator according to the determined control signal. The control unit 200 is configured to carry out a detection of the measurement signal, the determination of the control signal and the corresponding control of the stator 340 several times repeatedly in a sequence of control cycles. It is further provided that in each control cycle, a plurality of measurement points corresponding to the measurement signal recorded at a plurality of time points is taken into account by determining the control signal on the basis of the plurality of measurement points.

The control unit 200 is connected to the blood pump 300 by means of a driveline 305 for transmitting the measurement signal and the control signal.

The control unit 200 is preferably configured to determine the control signal in such a way that the rotor is adjusted into a target bearing position at which external forces acting on the rotor 320 along the bearing direction of action add up to a predefined force, in particular a zero force, and/or at which a power applied to generate the variable stator magnetic field is minimal (also referred to as zero-force control).

As may be inferred from the foregoing, the control unit 200 according to the examples described above is particularly suitable for carrying out a method for controlling the blood pump 300, wherein the method comprises:
determining, on the basis of the measurement signal, a control signal for varying the stator magnetic field, in such a way that the rotor 320 is supported in the housing 301 contact-free along the bearing direction of action by means of the bearing force and, by means of the torque, a rotation of the rotor 320 about the rotation axis X is generated, in particular open-loop- and/or closed-loop-controlled, wherein a specification for a rate of change of the rotation speed of the rotation of the rotor 320 about the rotation axis X is limited upwardly by a predefined maximum rate of change, such as to limit an influence of a change in the rotation speed on the determined control signal, and
controlling the stator 340 in accordance with the determined control signal.

The predefined maximum rate of change can be selected here such that the influence of the change in the rotation speed by the control of the stator 340 in accordance with the determined control signal on the control signal, in particular on a determination of the bearing position and/or bearing speed performed on the basis of the measurement signal and contributing to the control signal is limited compared to control of the stator 340 in accordance with a control signal that is determinable without limitation of the rate of change of the rotation speed to the predefined maximum rate of change. The control unit 200 is preferably configured to determine the control signal in such a way that an actual and/or measured rate of change of the rotation speed of the rotor does not reach or exceed the preset and/or the preset maximum rate of change when the stator is actuated in accordance with the determined control signal.

The control unit 200 is configured to estimate a state vector on the basis of the measurement signal, comprising a plurality of state parameters of a model of the rotation of the rotor 320, and to determine the control signal on the basis of the state vector. The estimation of the one or more state parameters of the model is carried out by means of an observer or by observation (in the sense of control engineering). The observer may take on various forms here.

In the following, exemplary pump controls are explained with reference to FIG. 3 to FIG. 5.

FIG. 3 shows in a first example a basic control loop of the pump control with observation, comprising a controller 501, a controlled system 502 (comprising the stator 340 and the rotor 320) and an observer 503. Based on a control deviation e, the controller 501 determines a manipulated variable vector u, which is fed to both the controlled system 502 and the observer 503. The physical response of the controlled system 502 determines the measured variable vector y, which is also fed to the observer 503. The observer estimates the state parameters of the state vector x, on the basis of which the control deviation e is determined as a deviation from the reference variable vector w.

In a second example of the pump control, the basic structure of which is also as shown in FIG. 3, the observer 503 is configured to estimate the state parameters of the state vector using an Extended Kalman Filter (EKF).

Such an EKF-based observer is based on the following equations:

$$\frac{dx}{dt} = f ( x ) + g ( x ) u , \qquad ( equation 5 a )$$

$$y = h ( x ) . \qquad ( equation 5 b )$$

Here, f(x) is a transition function which describes the change in state

$$\frac{dx}{dt}$$

as a function of the internal state x and g(x) is a control function which describes the change in state as a function of a manipulated variable vector u, h(x) is an observation function, wherein the functions f, g and h—unlike with a classic Kalman filter—do not have to be linear functions.

In the case of the blood pump 300 with back-EMF measurement, axial bearing and rotation control, the measured variable vector y contains the measured induction voltages, the manipulated variable vector u contains the torque M_{θ} and the bearing force F_{Z}:

$$y = [ \begin{matrix}{V}_{a} \\ {V}_{b}\end{matrix} ] , \qquad ( equation 6 a )$$

$$u = [ \begin{matrix}{M}_{θ} \\ {F}_{Z}\end{matrix} ] . \qquad ( equation 6 b )$$

The measurement equations for the measured induction voltages may be derived as follows. The magnetic field B (θ,z) of the rotor 320 in the axial direction on a circle with radius r at a distance z from the magnet surface is approximately given by

$$B ( θ , z ) = {B}_{0} sin ( θ ) {e}^{- z k} = {B}_{0} sin ( {n}_{p ρ z} {θ}_{mech} ) {e}^{- z κ} , \qquad ( equation 7 )$$

wherein B₀ is a magnetic field strength at z=0, θ is an electrical rotation angle, θ_{mech} is a mechanical rotation angle, n_{ppz} is the number of pole pairs of the BLDC motor and k is a wave number. The rotor 320 therefore generates a non-linear, in particular approximately exponentially decaying, permanent magnetic field along the bearing direction of action. The approximation applies to a sinusoidal magnetic field, which occurs in particular with ironless motors or motors with a large air gap. Blood pumps are often equipped with motors with a relatively large air gap, as fluid has to be pumped or at least bridged in the air gap. The method described may also be applied to non-sinusoidal magnetic fields, in particular trapezoidal magnetic field curves or magnetic curves consisting of a linear combination of several harmonic sinusoidal components.

With the definition B_{mag}=B₀e^{-zk}, the two-phase back-EMF may be represented as

$${B}_{a} = {B}_{mag} sin ( θ ) , \qquad ( equation 8 a )$$

$${B}_{b} = {B}_{mag} cos ( θ ) . \qquad ( equation 8 b )$$

Using the law of induction (equation 1), the following measurement equations are obtained:

$$\frac{{V}_{a}}{N A} = - ( \frac{d {B}_{m a g}}{dt} sin ( θ ) + {B}_{mag} \frac{d θ}{dt} cos ( θ ) ) , \qquad ( equation 9 a )$$

$$\frac{{V}_{b}}{N A} = - ( \frac{d {B}_{m a g}}{dt} cos ( θ ) - {B}_{mag} \frac{d θ}{dt} sin ( θ ) ) . \qquad ( equation 9 b )$$

Returning to the model of the EKF-based observer, the following entries may be assigned to the state vector s according to the dependencies contained in equations 7a and 7b:

$$x = [ \begin{matrix}{B}_{mag} \\ \frac{d {B}_{mag}}{dt} \\ θ \\ \frac{d θ}{dt}\end{matrix} ] . \qquad ( equation 10 )$$

Various influences such as manufacturing tolerances, temperature changes or changes in the viscosity of the fluid ensure that the system dynamics of the controlled system show an uncertainty. However, the system dynamics are explicitly required in the equations for the transfer of the manipulated variables to the angle and position of the rotor 320. If the mathematical model deviates from reality, large estimation errors may occur. This point therefore argues against taking the manipulated variables into account in the EKF. Another argument against this is that there are unmeasurable disturbances that affect the system and the effects of which must nevertheless be estimated by the EKF.

However, a distinction may be made between the manipulated variable torque and the manipulated variable bearing force. The dynamics of bearing force on bearing position are significantly more complex than the dynamics of torque on rotation angle, as the closed control loop must be taken into account. In addition, while the bearing force specification is typically zero (see discussion of zero force control above) and the controller only has to compensate for disturbances in this case, the torque specification is very large, especially at high speeds, and should also change quickly for rapid speed changes. For these reasons, the following system is also suitable for the EKF, in which only the torque is taken into account as a manipulated variable:

$$x = [ \begin{matrix}{B}_{mag} \\ \frac{d {B}_{mag}}{dt} \\ θ \\ \frac{d θ}{dt} \\ {K}_{M}\end{matrix} ] , \qquad ( equation 10 ‘ )$$

$$u = {M}_{θ} . \qquad ( equation 6 b ‘ )$$

Here, it is provided that the state vector comprises a torque amplification factor KM, which corresponds to a ratio of the rotation speed and the torque.

For the interpretation of an observer, in particular but not exclusively in the case of the EKF-based observer described as an example, the following considerations regarding the coupling between the estimation of the rotation speed and the estimation of the bearing speed should advantageously be taken into account.

The measurement equations (equations 7a, 7b) may also be written in the following form:

$${V}_{a} = - N A {B}_{0} {e}^{- kz} ( - k \dot{z} sin ( θ ) + \dot{θ} cos ( θ ) ) , \qquad ( equation 9 a ‘ )$$

$${V}_{b} = - NA {B}_{0} {e}^{- k z} ( - k \dot{z} cos ( θ ) - \dot{θ} sin ( θ ) ) . \qquad ( equation 9 b ‘ )$$

The time derivatives

$$\frac{d θ}{dt} and \frac{d z}{dt}$$

were displayed as θ and z respectively. The following assumptions are now made: the observer is converged, which means that z and θ are known; the estimated values of the rotation speed and the bearing speed are identical to the estimated values of the previous time step. The following apply:

$${θ}_{e} = θ , {\dot{θ}}_{e} = \dot{θ} + {\dot{θ}}_{c} , {z}_{e} = z , {\dot{z}}_{e} = \dot{z} + {\dot{z}}_{c} , \qquad ( equation 11 )$$

wherein the index e denotes the estimated values and the index c the deviation from the respective true value. If estimated values V_{a,e}, V_{b,e} for the induction voltages are calculated from the estimated values according to equations 9 by inserting them into equations 7a′, 7b′ and the difference is formed with the measured values, the deviations are as follows

$$Δ a = {V}_{a , e} - {V}_{a} = {NAB}_{0} {e}^{- k z} ( k {\dot{z}}_{c} sin ( θ ) - {θ}_{c} cos ( θ ) ) , \qquad ( equation 12 a )$$

$$Δ b = {V}_{b , e} - {V}_{b} = NA {B}_{0} {e}^{- k z} ( k {\dot{z}}_{c} cos ( θ ) + {\dot{θ}}_{c} sin ( θ ) ) , \qquad ( equation 12 b )$$

the correction to be made to the speeds (equal to the value of the deviation) may be represented as follows:

$${\dot{z}}_{c} = \frac{Δ a {sin}_{} ( θ ) + Δ b cos ( θ )}{k N A {B}_{0} {e}^{- kz}} , \qquad ( equation 13 a )$$

$${\dot{θ}}_{c} = \frac{Δ b sin ( θ ) - Δ a cos ( θ )}{{NAB}_{0} {e}^{- kz}} . \qquad ( equation 13 b )$$

If the sampling time is sufficiently short, the above assumptions and resulting approximations are justified. Nevertheless, a significant estimation error of the bearing position z remains if the rotation speed changes significantly, as the estimate is then based on a rotation speed of the previous time step that is no longer correct, which leads to an erroneous estimate of the angle.

The set-up must therefore take into account the maximum changes in bearing speed and the maximum changes in rotation speed to be expected between two time steps and how the corresponding estimation errors of the rotation speed and bearing speed affect the other variables to be estimated.

Omitting the assumption that the angle and bearing position are known (i.e. it is assumed that the angle and bearing position are also subject to estimation errors), a discrete-time equivalent of equations 9 may be given as follows:

$${θ}_{e} ( k ) = {θ}_{e} ( k - 1 ) + {\dot{θ}}_{e} ( k - 1 ) T , \qquad ( equation 14 a )$$

$${\dot{θ}}_{e} ( k ) = {\dot{θ}}_{e} ( k - 1 ) = \dot{θ} + {\dot{θ}}_{c} , \qquad ( equation 14 b )$$

$${z}_{e} ( k ) = {z}_{e} ( k - 1 ) + {\dot{z}}_{e} ( k - 1 ) T , \qquad ( equation 14 c )$$

$${\dot{z}}_{e} ( k ) = {\dot{z}}_{e} ( k - 1 ) = \dot{z} + {\dot{z}}_{c} . \qquad ( equation 14 d )$$

The estimated correction to be made to the bearing speed may then be obtained from this:

$${\dot{z}}_{c , e} = \frac{Δ {a}_{e} sin ( {θ}_{e} ) + Δ {b}_{e} cos ( {θ}_{e} )}{{kNAB}_{0} {e}^{- {kz}_{e}}} , \qquad ( equation 15 a )$$

wherein the following applies for Ade:

$$Δ {a}_{e} = - N A {B}_{0} {e}^{- kz} ( - k {\dot{z}}_{e} sin ( {θ}_{e} ) + {\dot{θ}}_{e} cos ( {θ}_{e} ) ) + N A {B}_{0} {e}^{- kz} ( - k \dot{z} sin ( θ ) + \dot{θ} cos ( θ ) ) \qquad ( equation 15 b )$$

(Ab, analog).

If the bearing speed is constant and known, i.e. z=zₑ=0 and ż=Żₑ=0 apply, the following applies:

$${\dot{z}}_{c , e} = \frac{\dot{θ}}{{kNAB}_{0}} sin ( {θ}_{e} - θ ) . \qquad ( equation 16 )$$

An estimation error of the rotor angle therefore leads to an error in the bearing speed. A rapid change in the rotation speed leads to larger estimation errors of the bearing speed with the same dynamics of the observer. Since large estimation errors of the bearing position inevitably lead to the rotor 320 hitting a wall of the housing 301 at some point, the rate of change of the rotor speed must be limited.

In the case of the EKF, a Q-matrix describing the system noise must be structured in such a way that the uncertain status parameters of bearing speed and rotation speed in particular are corrected quickly. A slower correction is sufficient for the remaining status parameters.

In a third example of pump control, the control unit 200 is configured to estimate the state parameters using a manually designed observer instead of the EKF. This should substantially approximate the behavior of the EKF-based observer, which in particular may reduce the required computational effort.

In the manual interpretation, an observer gain K is to be determined, which links an estimation error ye of the measured variable vector y in each time step with a state correction Δx of the state vector x:

$$Δ x = {Ky}_{e} . \qquad ( equation 17 )$$

The estimation task to be solved using the observer may be divided into three sub-tasks: 1. Adaptation of

$$\frac{{dB}_{mag}}{dt}$$

$$and$$

$$\frac{d θ}{dt} ;$$

2. Adaptation of B_{mag} and θ, wherein this adaptation is slower than the adaptation of

$$\frac{{dB}_{mag}}{dt}$$

$$and$$

$$\frac{d θ}{dt} ;$$

### 3. Estimation of K_{M}.

For each state parameter xᵢ, a maximum gain kᵢ may then be specified with which the adaptation is made:

$$\begin{matrix}Δ {x}_{i} = {\sum }_{j = 1}^{2} {k}_{i} {e}_{ij} {y}_{e , j} , & 0 < {e}_{ij} \leq 1.\end{matrix} \qquad ( equation 18 )$$

Two alternative methods are proposed for determining the scaling values eᵢⱼ contained in equation 16.

In the first method, the eᵢⱼ are determined in advance from the measured variables yᵢ. For this purpose, a complex number Y and a normalized complex number F are defined using the measured induction voltages:

$$Y = {V}_{a} + i {V}_{b} , \qquad ( equation 19 a )$$

$$F = \frac{Y}{\vert Y \vert} . \qquad ( equation 19 b )$$

Based on an empirically selected specification of complex numbers fᵢⱼ, the eᵢⱼ may then be calculated as

$${e}_{ij} = real ( {f}_{ij} F ) . \qquad ( equation 20 )$$

In the second method for determining the eᵢⱼ, the eᵢⱼ are optimized separately according to the above sub-tasks. For example, a basis may be predefined for the optimization in the first sub-task, for example the basis

$$[ \begin{matrix}{e}_{2} \\ {e}_{4}\end{matrix} ] = [ \begin{matrix}sin ( \frac{2 π k}{N} ) \\ cos ( \frac{2 π k}{N} )\end{matrix} ] , k ∈ \{ 1 , 2 , … , N \} \qquad ( equation 21 )$$

and then in each step a k may be selected that minimizes the measurement error. A suitable choice of the number N of basis vectors may be made to achieve the desired compromise between computational effort and estimation accuracy.

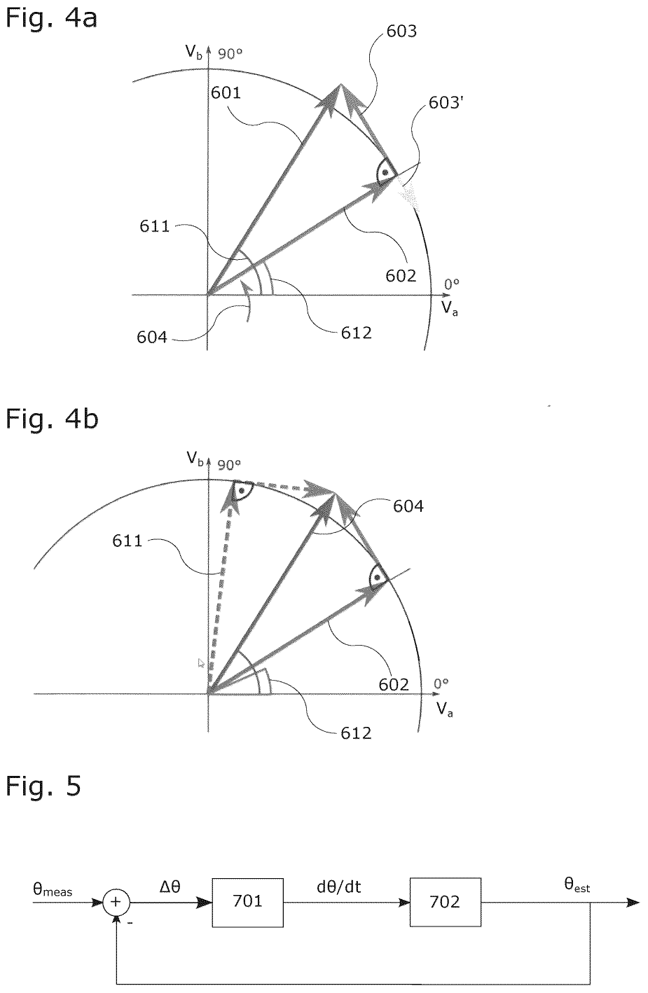

In a fourth example of pump control, illustrated in FIG. 4a, FIG. 4b and FIG. 5, the control unit 200 is configured to estimate the state parameters in a particularly simple way using a vector decomposition of the measured back-EMF into a rotation component and a bearing position component.

FIG. 4a shows a pointer diagram for an induced back-EMF, represented as a back-EMF vector 601 in a two-phase representation, wherein this representation may be generated in particular by means of d/q transformation from a three-phase measurable back-EMF. The back-EMF vector may be broken down into a rotation component 602 and a perpendicular bearing position component 603, i.e. represented as the vectorial sum of these two components.

The value of the rotation component 602 is

$$NA {B}_{mag} \frac{d θ}{dt} ,$$

i.e. in particular proportional to the speed of the rotor 320. The phase angle 604 of the rotation component 602 corresponds to the electrical rotation angle. The rotation component 602 rotates in the pointer diagram according to the electrical rotor speed (rotation direction 605). The bearing position component 603 is perpendicular to the rotation component 602 and has the value

$$NA \frac{{dB}_{mag}}{dt}$$

and is therefore proportional to the translational rotor movement. The induction voltage measurable at the terminals of the coils of the motor coil arrangement 343 corresponds to the value of the back-EMF vector 601. Since this may be represented as the vectorial sum of the rotation component 602 and the bearing position component 603, the following expression may be read off:

$$\sqrt{\frac{1}{{( NA )}^{2}} ( {V}_{a}^{2} + {V}_{b}^{2} ) - {{B}_{mag}^{2} ( \frac{d θ}{dt} )}^{2}} = \vert \frac{{dB}_{mag}}{dt} \vert . \qquad ( equation 22 )$$

This relationship only gives the magnitude, but not the sign of the magnetic field change. Both constellations shown in FIG. 4b are therefore possible (dashed and solid arrows). To determine the sign, an estimate of the electrical rotation angle θ is also required.

For this purpose, the decoupled dynamics of bearing position control and rotation control according to the application may be utilized on the one hand, and on the other hand the fact that

$$\frac{d {β}_{mag}}{dt}$$

is zero on average over time, since the rotor 320 may only remain within a limited deflection range along the bearing direction of action. It is therefore possible to estimate the rotation speed and the rotation angle using a simple PID control loop comprising a PID controller 701 and an integrator 702, which is shown in FIG. 5.

The sign of

$$\frac{d {β}_{mag}}{dt}$$

may men be determined as shown in FIG. 4b by ascertaining which of the two possible constellations is closer to the estimated rotation angle 606 (θₑₛₜ). Furthermore, since the rotation component 602 is perpendicular to the bearing position component 603, the magnetic field change may ultimately be calculated as

$$\frac{{dB}_{mag}}{dt} = - \frac{1}{NA} [ sin ( {θ}_{est} ) {V}_{a} + cos ( {θ}_{est} ) {V}_{b} ] . \qquad ( equation 23 )$$

Regardless of how the pump control is implemented, the control unit 200 may also be configured

to detect a plurality of measurement points, corresponding to at least a component of the measurement signal at a plurality of time points with constant rotation speed and constant bearing position of the rotor 320,

to fit a periodic correction function, in particular a sine function or a plurality of superposed sine functions, to at least one component of the measurement signal by means of optimization.

Preferably, a corresponding periodic correction function is fitted to each induction voltage of the multi-dimensional back-EMF measurement signal. The required constant axial position may, for example, be adjusted using an additional sensor, such as a Hall sensor. In the example described here, a sinusoidal function with a common amplitude A, angular frequency ω and phase ϕ0 is fitted to each induction voltage, wherein the sinusoidal functions differ by a fixed phase offset corresponding to a number of coil pairs of the motor coil arrangement 343. With three pairs of coils and corresponding induction voltages VA, VB, VC, the phase offset is 120°. In this case, a figure of merit to be minimized for the optimization is:

$$I = {\sum }_{k = 0}^{N} {( A sin ( ω kT + {φ}_{0} ) - {V}_{A} ( k ) )}^{2} + {( A sin ( ω kT + {φ}_{0} + \frac{2}{3} π ) - {V}_{B} ( k ) )}^{2} + {( A sin ( ω kT + {φ}_{0} + \frac{4}{3} π ) - {V}_{C} ( k ) )}^{2} . \qquad ( equation 24 )$$

In this example, the control unit 200 is configured to compensate, on the basis of the correction function fitted in this way, a disturbance of the measurement signal that depends on the rotation angle of the rotor, in particular one that occurs periodically with a period of rotation of the rotor, so that its influence on the control signal is reduced.

As mentioned above, the (measurable) back-EMF 601 is composed of a rotation component 602 and a bearing position component 603. The rotation component 602 is decisive for optimum commutation, as this may be used to determine the actual rotation angle of the rotor. The disturbing bearing position component 603 depends on the geometry-dependent constants NA and on the rate of change of the magnetic field amount

$$\frac{{dB}_{mag}}{dt} .$$

The rate of change of the magnetic field amount in turn depends on the course of the axial position. If the axial rotor position (bearing position) or the axial rotor speed (bearing speed) is measured with a sensor or otherwise determined, the rotation component 602 may be calculated from the back-EMF 601 and the bearing position component 603 and thus may be commutated more precisely using the back-EMF. FIG. 4a shows the geometric relationship between the back-EMF components. The rotation component 602 may be clearly determined from the condition that the bearing position component 603 is perpendicular to the rotation component 602.

In order to enable such a determination, the blood pump 300 may, for example, comprise a rotor position sensor by means of which a sensor signal is provided depending on the bearing position and/or bearing speed of the movement of the rotor 320 along the bearing direction of action. The control unit 200 may—in addition or as an alternative to the above-described limiting of the rotation speed rate of change—be configured to receive this sensor signal and, on the basis of the sensor signal and the measurement signal, to determine a corrected measurement signal which corresponds to the component of the measurement signal which is dependent on the rotation angle and/or the rotation speed of the rotation of the rotor 320 about the rotation axis X. In particular, to determine the corrected measurement signal, the rotation component 602 may be determined as described in the previous paragraph.

The control unit 200 may be configured to determine the rotary component of the control signal for closed-loop control of the rotation of the rotor 320 on the basis of the corrected measurement signal.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one or more element alone or the one or more element in combination with one or more of the other elements which may also include, in combination, additional elements not listed. Unless otherwise indicated or the context suggests otherwise, as used herein, “a” or “an” means “at least one” or “one or more.”

The application also relates to the following aspects:

A control unit (200) for a blood pump (300),
wherein the blood pump (300) comprises a rotor (320) magnetically supported in a housing (301) and rotatable about a rotation axis (X) for conveying a fluid, and a stator (340), wherein the stator (340) is configured to generate a variable stator magnetic field for exerting on the rotor (320) a variable bearing force along a bearing direction of action and a torque about the rotation axis (X);
wherein the blood pump (300) is configured to provide a measurement signal comprising a component dependent on a bearing position and/or bearing speed of a movement of the rotor (320) along the bearing direction of action and a component dependent on a rotation angle and/or a rotation speed of a rotation of the rotor (320) about the rotation axis (X);
wherein the control unit (200) is configured
to determine, on the basis of the measurement signal, a control signal for varying the stator magnetic field, in such a way that the rotor (320) is supported in the housing (301) contact-free along the bearing direction of action by means of the bearing force and, by means of the torque, a rotation of the rotor (320) about the rotation axis (X) is generated, in particular open-loop- and/or closed-loop-controlled, wherein a specification for a rate of change of the rotation speed of the rotation of the rotor (320) about the rotation axis (X) is limited upwardly by a predefined maximum rate of change, such as to limit an influence of a change in the rotation speed on the determined control signal, and
to control the stator (340) in accordance with the determined control signal.

The control unit (200) according to aspect 1, wherein the predefined maximum rate of change is selected such that the influence of the change in the rotation speed by the control of the stator (340) in accordance with the determined control signal on the control signal, in particular on a determination of the bearing position and/or bearing speed performed on the basis of the measurement signal and contributing to the control signal is limited compared to control of the stator (340) in accordance with a control signal that is determinable without limitation of the rate of change of the rotation speed to the predefined maximum rate of change.

The control unit (200) according to any one of the preceding aspects, wherein the predefined maximum rate of change is proportional to a dynamic parameter, the movement of the rotor (320) along the bearing direction of action, wherein the dynamic parameter is, in particular, a parameter of an open-loop or closed-loop control of the movement of the rotor (320) along the bearing direction of action and/or a variable determined on the basis of the measurement signal.

The control unit (200) according to aspect 3, wherein the dynamic parameter is a bearing acceleration and/or deflection frequency and/or deflection frequency rate of change of the movement of the rotor (320) along the bearing direction of action and/or a gain crossover frequency and/or resonance frequency of a closed-loop control of the movement of the rotor along the bearing direction of action.

The control unit (200) according to any one of the preceding aspects, wherein the predefined maximum rate of change is given by a time constant, in particular a delay time and/or step response time and/or rise time of a closed-loop control of the rotation of the rotor (320).

The control unit (200) according to any one of the preceding aspects, wherein the predefined maximum rate of change is at most half, preferably at most a fifth, in particular at most a tenth of a characteristic rate of change of the movement of the rotor (320) along the bearing direction of action, and/or wherein a characteristic time constant, by which the predefined maximum rate of change is given, is at least twice, preferably five times, in particular at least ten times a characteristic time constant of the movement of the rotor (320) along the bearing direction of action.

The control signal (200) according to any one of the preceding aspects, wherein the measurement signal corresponds to a back-electromotive force (back-EMF) induced on account of a magnetic field change in the stator (340) caused by the rotation of the rotor (320) and/or the movement of the rotor (320) along the bearing direction of action.

The control unit (200) according to any one of the preceding aspects, configured
to estimate, on the basis of the measurement signal, one or more state parameters of a model of the movement of the rotor (320), in particular the rotation of the rotor (320) about the rotation axis and/or the movement of the rotor (320) along the bearing direction of action, wherein the state parameter or state parameters comprise in particular the rotation angle and/or the rotation speed and/or the rate of change of the rotation speed and/or a rotation frequency and/or a rotation frequency rate of change of the rotation of the rotor (320) and/or the bearing position and/or the bearing speed and/or a bearing acceleration and/or a deflection frequency and/or a deflection frequency rate of change of the movement of the rotor (320) along the bearing direction of action and/or the bearing force and/or the torque and/or a field strength of a magnetic field generated within the stator (340) by the rotor (320) and/or a change of this field strength over time, and
to determine the control signal on the basis of one or more of the estimated state parameters.

The control unit (200) according to aspect 8, wherein the state parameter or state parameters comprise a torque amplification factor, which corresponds to a ratio of the rotation speed and the torque.

The control unit (200) according to aspect 8 or 9, configured to estimate the state parameter or state parameters using an extended Kalman filter.

The control unit (200) according to any one of aspects 8 to 10, configured to estimate the state parameter or state parameters using a manually configured observer and/or using a vector decomposition of a measured back-EMF into a rotation component (602) and a bearing position component (603).

The control unit (200) according to any one of aspects 8 11, wherein the control unit (200) is configured to determine the control signal such that the rotor (320) is adjusted into a target bearing position at which external forces acting on the rotor (320) along the bearing direction of action add up to a predefined force, in particular a zero force, and/or at which a power applied to generate the variable stator magnetic field is minimal.

The control unit (200) according to any one of aspects 8 12, wherein a plurality of measurement points, corresponding to the measurement signal at a plurality of time points, is detected and the control unit (200) is configured to determine the control signal on the basis of the plurality of measurement points.

The control unit (200) according to any one of aspects 8 to 13, further configured
to detect a plurality of measurement points, corresponding to at least a component of the measurement signal at a plurality of time points with constant rotation speed and known, in particular constant, bearing position of the rotor (320),
to fit a periodic correction function, in particular a sine function or a plurality of superposed sine functions, to at least one component of the measurement signal by means of optimization.

A pump system, comprising
a blood pump (300),
wherein the blood pump (300) comprises a rotor (320) magnetically supported in a housing (301) and rotatable about a rotation axis (X) for conveying a fluid, and a stator (340), wherein the stator (340) is configured to generate a variable stator magnetic field for exerting on the rotor (320) a variable bearing force along a bearing direction of action and a torque about the rotation axis (X);
wherein the blood pump (300) is configured to provide a measurement signal comprising a component dependent on a bearing position and/or speed of a movement of the rotor (320) along the bearing direction of action and a component dependent on a rotation angle and/or a rotation speed of a rotation of the rotor (320) about the rotation axis (X); and
a control unit (200) according to any one of aspects 8 to 14, configured to control the blood pump (300).

The pump system according to aspect 15, wherein the rotor (320) and the stator (340) form an axial flux motor or radial flux motor and the bearing direction of action runs substantially parallel to the rotation axis (X).

The pump system according to aspect 15 or 16, wherein the rotor (320) and the stator (340) form a radial flux motor and the bearing direction of action is in a radial direction at an angle different from zero, in particular substantially perpendicular, to the rotation axis (X) of the rotor (320), wherein the rotor (320), in addition to the aforementioned bearing direction of action, is preferably supported by active magnetic bearing in the housing (301) also along a second bearing direction of action.

The pump system according to any one of aspects 15 to 17, wherein the rotor (320) generates a permanent magnetic field decaying non-linearly, in particular at least approximately exponentially, along the bearing direction of action and/or the rotation axis (X).

A method for controlling a blood pump (300),
wherein the blood pump (300) comprises a rotor (320) magnetically supported in a housing (301) and rotatable about a rotation axis (X) for conveying a fluid, and a stator (340), wherein the stator (340) is configured to generate a variable stator magnetic field for exerting a variable bearing force along a bearing direction of action along the bearing direction of action;
wherein the blood pump (300) is configured to provide a measurement signal comprising a component dependent on a bearing position and/or speed of a movement of the rotor (320) along the bearing direction of action and a component dependent on a rotation angle and/or a rotation speed of a rotation of the rotor (320) about the rotation axis (X);
wherein the method comprises:
determining, on the basis of the measurement signal, a control signal for varying the stator magnetic field, in such a way that the rotor (320) is supported in the housing (301) contact-free along the bearing direction of action by means of the bearing force and, by means of the torque, a rotation of the rotor (320) about the rotation axis (X) is generated, in particular open-loop- and/or closed-loop-controlled, wherein a specification for a rate of change of the rotation speed of the rotation of the rotor (320) about the rotation axis (X) is limited upwardly by a predefined maximum rate of change, such as to limit an influence of a change in the rotation speed on the determined control signal, and controlling the stator (340) in accordance with the determined control signal.

### LIST OF REFERENCE SIGNS

100 pump system,
200 control unit,
300 blood pump,
301 housing,
302 inlet,
303 outlet,
304 volute,
305 driveline,
320 rotor,
321 blading,
322 Inlet-side rotor magnet bearing,
323 outlet-side rotor magnet bearing,
324 motor magnet arrangement,
340 stator,
341 inlet-side stator magnet bearing,
342 outlet-side stator magnet bearing,
343 motor coil arrangement,
344 control coil,
401, 402, 403, 404 measurement signal curve,
501 controller,
502 controlled system,
503 observer,
60 back-EMF vector,
602 rotation component,
603 bearing position component,
604 phase angle,
605 rotation direction,
606 estimated rotation angle,
701 PID controller,
702 integrator,
X rotation axis.

1. A control unit for a blood pump
comprising a rotor magnetically supported in a housing and rotatable about a rotation axis for conveying a fluid, and a stator configured to generate a variable stator magnetic field for exerting on the rotor a variable bearing force along a bearing direction of action and a torque about the rotation axis;
wherein the blood pump is configured to provide a measurement signal comprising a component dependent on at least one of a bearing position and a bearing speed of a movement of the rotor along the bearing direction of action, and a component dependent on at least one of a rotation angle and/or a rotation speed of a rotation of the rotor about the rotation axis;
wherein the control unit is configured
to determine, on the basis of the measurement signal, a control signal for varying the stator magnetic field in such a way that the rotor is supported in the housing contact-free along the bearing direction of action by means of the bearing force and, by means of the torque, a rotation of the rotor about the rotation axis is generated, in particular open-loop- and/or closed-loop-controlled, wherein a specification for a rate of change of the rotation speed of the rotation of the rotor about the rotation axis is limited upwardly by a predefined maximum rate of change, such as to limit an influence of a change in the rotation speed on the determined control signal, and
to control the stator in accordance with the determined control signal.

2. The control unit of claim 1, wherein the predefined maximum rate of change is selected such that the influence of the change in the rotation speed by the control of the stator in accordance with the determined control signal on the control signal, in particular on a determination of at least one of the bearing position and bearing speed performed on the basis of the measurement signal and contributing to the control signal is limited compared to control of the stator in accordance with a control signal that is determinable without limitation of the rate of change of the rotation speed to the predefined maximum rate of change.

3. The control unit of claim 1, wherein the predefined maximum rate of change is proportional to a dynamic parameter of the movement of the rotor along the bearing direction of action, wherein the dynamic parameter is at least one of a parameter of an open-loop or closed-loop control of the movement of the rotor-along the bearing direction of action and a variable determined on the basis of the measurement signal.

4. The control unit of claim 3, wherein the dynamic parameter is at least one of a bearing acceleration frequency, a deflection frequency, a deflection frequency rate of change of the movement of the rotor along the bearing direction of action, a gain crossover frequency, and a resonance frequency of a closed-loop control of the movement of the rotor along the bearing direction of action.

5. The control unit of claim 1, wherein the predefined maximum rate of change is given by a time constant comprising at least one of a delay time, a step response time, and a rise time of a closed-loop control of the rotation of the rotor.

6. The control unit of claim 1, wherein at least one of:
the predefined maximum rate of change is at most half, at most a fifth, or at most a tenth of a characteristic rate of change of the movement of the rotor along the bearing direction of action, and
a characteristic time constant, by which the predefined maximum rate of change is given, is at least twice, at least five times, or at least ten times a characteristic time constant of the movement of the rotor along the bearing direction of action.

7. The control unit of claim 1, configured
to receive a sensor signal provided using a rotor position sensor of the blood pump, the sensor signal depending on at least one of the bearing position and bearing speed of the movement of the rotor along the bearing direction of action,
to determine, on the basis of the sensor signal and the measurement signal, a corrected measurement signal which corresponds to the component of the measurement signal dependent on at least one of the rotation angle and the rotation speed of the rotation of the rotor about the rotation axis, and
to determine a rotary component of the control signal for closed-loop control of the rotation of the rotor on the basis of the corrected measurement signal.

8. A control unit for a blood pump
comprising a rotor magnetically supported in a housing and rotatable about a rotation axis for conveying a fluid, and a stator configured to generate a variable stator magnetic field for exerting on the rotor a variable bearing force along a bearing direction of action and a torque about the rotation axis;
wherein the blood pump is configured to provide a measurement signal comprising a component dependent on at least one of a bearing position and a bearing speed of a movement of the rotor along the bearing direction of action, and a component dependent on at least one of a rotation angle and a rotation speed of a rotation of the rotor about the rotation axis;
wherein the blood pump comprises a rotor position sensor which is configured to provide a position sensor signal dependent on at least one of the bearing position and bearing speed of the movement of the rotor along the bearing direction of action;
wherein the control unit is configured
to determine, on the basis of the position sensor signal and the measurement signal, a corrected measurement signal which corresponds to the component of the measurement signal dependent on at least one of the rotation angle and the rotation speed of the rotation of the rotor about the rotation axis,
to determine, on the basis of the measurement signal, a control signal for varying the stator magnetic field, in such a way that the rotor is supported in the housing contact-free along the bearing direction of action by means of the bearing force and, by means of the torque, a rotation of the rotor about the rotation axis is generated, in particular open-loop- and/or closed-loop-controlled,
to determine, on the basis of the sensor signal and the measurement signal, a corrected measurement signal which corresponds to the component of the measurement signal dependent on at least one of the rotation angle and the rotation speed of the rotation of the rotor about the rotation axis,
wherein a rotary component of the control signal for closed-loop control of the rotation of the rotor on the basis of the corrected measurement signal is determined, and
to control the stator in accordance with the determined control signal.

9. The control unit of claim 8, wherein the measurement signal corresponds to a back-electromotive force induced on account of a magnetic field change in the stator caused by at least one of the rotation of the rotor and the movement of the rotor along the bearing direction of action.

10. The control unit of claim 8, configured
to estimate, on the basis of the measurement signal, one or more state parameters of a model of the movement of the rotor, wherein the one or more state parameters comprises at least one of the rotation angle, the rotation speed, the rate of change of the rotation speed, a rotation frequency of the rotation of the rotor, a rotation frequency rate of change of the rotation of the rotor, the bearing position, the bearing speed, a bearing acceleration, a deflection frequency of the movement of the rotor along the bearing direction of action, a deflection frequency rate of change of the movement of the rotor along the bearing direction of action, the bearing force, the torque, a field strength of a magnetic field generated within the stator by the rotor, and a change of this field strength over time, and
to determine the control signal on the basis of one or more of the estimated state parameters.

11. (canceled)

12. (canceled)

13. (canceled)

14. The control unit of claim 8, configured to determine the control signal such that the rotor is adjusted into a target bearing position at which external forces acting on the rotor along the bearing direction of action add up to at least one of a predefined zero force, and at which a power applied to generate the variable stator magnetic field is minimal.

15. The control unit of claim 8, wherein a plurality of measurement points, corresponding to the measurement signal at a plurality of time points, is detected and the control unit is configured to determine the control signal on the basis of the plurality of measurement points.

16. The control unit of claim 8, further configured
to detect a plurality of measurement points, corresponding to at least a component of the measurement signal at a plurality of time points with constant rotation speed and known, in particular constant, bearing position of the rotor,
to fit a periodic correction function comprising a sine function or a plurality of superposed sine functions, to at least one component of the measurement signal by means of optimization.

17. A pump system, comprising:
a blood pump (300);
comprising a rotor magnetically supported in a housing and rotatable about a rotation axis for conveying a fluid, and a stator configured to generate a variable stator magnetic field for exerting on the rotor a variable bearing force along a bearing direction of action and a torque about the rotation axis;
wherein the blood pump is configured to provide a measurement signal comprising a component dependent on at least one of a bearing position and speed of a movement of the rotor along the bearing direction of action, and a component dependent on at least one of a rotation angle and a rotation speed of a rotation of the rotor about the rotation axis; and
a control unit of claim 8, configured to control the blood pump.

18. The pump system of claim 17, wherein the rotor and the stator form an axial flux motor or radial flux motor and the bearing direction of action runs substantially parallel to the rotation axis.

19. The pump of claim 17, wherein the rotor and the stator form a radial flux motor and the bearing direction of action is in a radial direction at an angle different from zero, in particular substantially perpendicular, to the rotation axis of the rotor, wherein the rotor further is supported by active magnetic bearing in the housing along a second bearing direction of action.

20. The pump system of claim 17, wherein the rotor generates a permanent magnetic field decaying non-linearly, and at least approximately exponentially, along at least one of the bearing direction of action and the rotation axis.

21. A method for controlling a blood pump
comprising a rotor magnetically supported in a housing and rotatable about a rotation axis for conveying a fluid, and a stator configured to generate a variable stator magnetic field for exerting a variable bearing force along a bearing direction of action along the bearing direction of action;
wherein the blood pump is configured to provide a measurement signal comprising a component dependent on at least one of a bearing position and speed of a movement of the rotor along the bearing direction of action, and a component dependent on at least one of a rotation angle and a rotation speed of a rotation of the rotor about the rotation axis;
wherein the method comprises:
determining, on the basis of the measurement signal, a control signal for varying the stator magnetic field, in such a way that the rotor is supported in the housing contact-free along the bearing direction of action by means of the bearing force and, by means of the torque, a rotation of the rotor about the rotation axis is generated, in particular open-loop- and/or closed-loop-controlled, wherein a specification for a rate of change of the rotation speed of the rotation of the rotor about the rotation axis is limited upwardly by a predefined maximum rate of change, such as to limit an influence of a change in the rotation speed on the determined control signal, and
controlling the stator in accordance with the determined control signal.

22. A method for controlling a blood pump
comprising a rotor magnetically supported in a housing and rotatable about a rotation axis for conveying a fluid, and a stator configured to generate a variable stator magnetic field for exerting a variable bearing force along a bearing direction of action along the bearing direction of action;
wherein the blood pump is configured to provide a measurement signal comprising a component dependent on at least one of a bearing position and speed of a movement of the rotor along the bearing direction of action, and a component dependent on at least one of a rotation angle and a rotation speed of a rotation of the rotor about the rotation axis;
wherein the blood pump comprises a rotor position sensor which is configured to provide a position sensor signal dependent on at least one of the bearing position and bearing speed of the movement of the rotor along the bearing direction of action;
wherein the method comprises:
determining, on the basis of the position sensor signal and the measurement signal, a corrected measurement signal which corresponds to the component of the measurement signal dependent on at least one of the rotation angle and the rotation speed of the rotation of the rotor about the rotation axis,
determining, on the basis of the corrected measurement signal, a control signal for varying the stator magnetic field, in such a way that the rotor is supported in the housing contact-free along the bearing direction of action by means of the bearing force and, by means of the torque, a rotation of the rotor about the rotation axis is generated, in particular open-loop- and/or closed-loop-controlled, and
determining, on the basis of the sensor signal and the measurement signal, a corrected measurement signal which corresponds to the component of the measurement signal dependent on at least one of the rotation angle and the rotation speed of the rotation of the rotor about the rotation axis,
wherein a rotary component of the control signal for closed-loop control of the rotation of the rotor on the basis of the corrected measurement signal is determined, and
controlling the stator in accordance with the determined control signal.

23. The control unit of claim 10, comprising one or more of:
the one or more state parameters comprises a torque amplification factor, which corresponds to a ratio of the rotation speed and the torque;
the control unit is configured to estimate the one or more state parameters using an extended Kalman filter; and
the control unit is configured to estimate the one or more state parameters using at least one of a manually configured observer and a vector decomposition of a measured back-electromotive force into a rotation component and a bearing position component.
